(19) United States
(12) Patent Application Publication
(10) Pub. No.: US 2023/0181464 A1
(43) Pub. Date: Jun. 15, 2023

(54) COMPOSITIONS FOR DELIVERY OF BIOACTIVE AGENTS INTO HAIR FOLLICLES

(71) Applicant: FOLLICLE PHARMA LTD, Rehovot (IL)

(72) Inventors: Doron FRIEDMAN, Carme-Yosef (IL); Tal BERMAN, Rishon-LeZion (IL); Inbal ZIV, Gedera (IL); Oron ZEEVI, Carme-Yosef (IL)

(21) Appl. No.: 17/924,859

(22) PCT Filed: May 12, 2021
(86) PCT No.: PCT/IL2021/050549

§ 371 Date: Nov. 11, 2022

Related U.S. Application Data
(60) Provisional application No. 63/024,085, filed on May 13, 2020.

(51) Int. Cl. A61K 9/107; A61K 31/58; A61K 47/44; A61P 17/14; A61Q 7/00; A61K 8/63

(52) U.S. Cl. CPC A61K 9/1075; A61K 31/58; A61K 47/44; A61P 17/14; A61Q 7/00; A61K 8/63; A61K 2800/21

(57) ABSTRACT

The present invention is directed to a composition for the delivery of bioactive agents into hair follicles. The composition is prepared as an oil-in-water emulsion, and comprises one or more lipophilic bioactive agents, one or more oil solvents, one or more emulsifiers and water, wherein said bioactive agents are substantially dissolved in the internal oil phase of said emulsion, and wherein the mean droplet size of said emulsion is in the range of about 200 to about 1000 nm. The invention also encompasses methods for delivering said compositions into the hair follicle, and for treating diseases and disorders thereof.

### BACKGROUND OF THE INVENTION

The structure from which each hair grows across the skin is called a follicle. Skin Follicle diseases include infectious diseases, immunological disorders, autoimmune diseases, blockage of sebaceous gland or of all hair follicle, cancers, and multiple cause inflammatory conditions. The follicles occupy only 0.2% to 2% of the skin area. Therefore, systemic or topical medications aimed at the follicles for treating a follicle disease or for cosmetic use are tremendously wasteful of the product, since the fraction of the dose that reaches the follicles is extremely small.

There is a need for efficient targeting of bioactive agents to the hair follicle for medication and for cosmetic and veterinary uses. There is a need to provide adequate drug level to the hair follicle while sparing the whole body or the whole skin from high doses and potential unwanted adverse effects and toxicity.

Many drugs or cosmetics that exert their pharmacological effects in the hair follicle often have side effects and may cause systemic toxicity or local irritation. Consequently, a pressing need exists for a targeted delivery system to the hair follicle which will reduce the systemic exposure and/or the topical amount delivered to the rest of the skin. Such bioactive agents may include for example: 5 alpha reductase inhibitors, Janus Kinase inhibitors, vitamin A derivatives, antibiotics, anti-inflammatory agents, antiparasitic agents, immune modulating agents, anesthetics and antioxidants and other topical cosmeceuticals such as benzoyl peroxide, azelaic acid, vitamin A and derivatives thereof.

U.S. Pat. No. 9,186,324 describe anhydrous emulsions for follicular delivery of drugs. However, these compositions are heavy and cause a sticky and greasy skin feeling and are not suitable, for example, for alopecia, or for facial or large body surface treatment. Since patient compliance is critical for clinical efficacy and success, a patient-friendly non-sticky, non-greasy and non-shiny product with good spreadability and fast absorption is anticipated by the customer and or patient as well as by the physicians.

US patent application 20200147071 discloses a method for stimulating the hair growth on the scalp of a human subject; however, it fails to show specific intrafollicular delivery or any targeting of drugs to the hair follicle or disclose any data showing reduced blood levels, or reduced side effects or reduced skin drug concentration in comparison to hair follicles.

Various publications disclose the use of liposomes and nano-liposomes as well as solid lipid nanoparticles and polymeric nanoparticles in the delivery of drugs to the hair follicle. However, the liposomes' encapsulation capacity for insoluble drugs is very limited. Furthermore, in many cases cause liposomes instability over time, and upscaling and production require the use of special equipment and processes.

Solid lipid nanoparticles are made either of polymers or fully saturated lipids that are solid at room temperature and thus are very limited in solubilizing hydrophobic drugs by their highly non-polar nature.

In sharp contrast to liposomes and solid lipid delivery systems, the novel compositions accommodate high strength of insoluble hydrophobic drugs, specifically deliver the drug into the hair follicle, are also highly stable over shelf-life period, and show very good skin feeling and user experience.

It is therefore one objective of the present invention to provide a pharmaceutical composition that permits the specific delivery of bioactive agent into the hair follicle organ of mammal skin, thereby resulting in significantly reduced systemic blood levels, as compared with prior-art compositions.

It is one objective of the present invention to provide a pharmaceutical composition that permits the specific delivery of bioactive agent into the hair follicle organ of mammal skin, thereby resulting in significantly reduced systemic blood levels, as compared with prior-art compositions.

It is another objective of the present invention to provide a composition that is capable of causing the specific delivery of one or more bioactive agents into the hair follicle organ and with much reduced whole skin exposure to the composition and with reduced local irritation.

It is a further objective of the invention to provide an effective composition which has significantly improved safety and patient tolerability when administered for hair follicle diseases, in comparison with oral and/or parenteral administration or conventional topical medications.

Further objectives and advantages of the composition of the present invention will become apparent as the description proceeds.

### SUMMARY OF THE INVENTION

According to the present invention there is provided a composition comprising at least one bioactive agent for delivery into the hair follicle (i.e. intrafollicular delivery), wherein the bioactive agent is dissolved in the internal oil phase of an oil-in-water emulsion and wherein the emulsion has a mean droplet size of about 200 to about 1,000 nanometer and the mean droplet size does not change significantly over the shelf life of the product, and wherein said composition exhibits physical and chemical stability for at least 6 months at accelerated storage conditions.

We have unexpectedly discovered that compositions comprising a bioactive agent that is dissolved in the oily phase of an oil-in-water emulsion with mean droplet size of about 200 nm to about 1,000 nm, and applied topically to the skin with slight rubbing, delivers a much greater dose fraction of the bioactive agent(s) into the follicle (i.e., intrafollicular delivery) as compared to the adjacent treated skin. We also found that a droplet size of about 400 nm to about 800 nm is preferable.

Micro and nano droplets tend to aggregate and coalesce upon storage due to a very large interface area. It has been unexpectedly discovered that specific compositions are stable for long time under shelf life and under accelerated stability conditions and their mean droplet size does not change or increase significantly.

In a preferred embodiment of the invention, the bioactive agent is preferably delivered into the hair follicles and does not significantly penetrate into the blood.

In a preferred embodiment of the invention, the topical product has a pleasant skin feeling and is non-greasy, non-sticky, non-shiny with fast absorption and good spreadability, and is suitable for use on hairy skin, scalp and face, on large exposed and on non-exposed (covered with clothes) skin surfaces.

The present invention is therefore primarily directed to a therapeutic composition, intended for use in the targeted delivery of bioactive agents into hair follicles. The composition is prepared in the form of an oil-in-water emulsion, comprising one or more lipophilic bioactive agents, one or more oil solvents, one or more emulsifiers and water; wherein said one or more bioactive agent is substantially dissolved in the internal oil phase of said emulsion;
wherein the mean droplet size of said emulsion is in the range of about 200 to about 1000 nm;
wherein the one or more oil solvents is soluble or miscible with ethanol;
wherein said one or more oil solvents are selected from the group consisting of a natural or synthetic unsaturated triglycerides, fatty acids, fatty alcohols, fatty esters or fatty ethers thereof;
and wherein said one or more oil solvents have a melting point of less than about 15° C.

In one preferred embodiment of this aspect of the invention, the above-disclosed composition further comprises at least one solubility enhancer.

In some preferred embodiments of this aspect of the present invention, the composition further comprises ethanol.

In some preferred embodiments, the droplet size of the oil-in-water emulsion is in the range of about 400 to about 800 nm.

As disclosed hereinabove, the one or more bioactive agents present in the composition of the prevent invention are lipophilic agents (preferably pharmaceutical, cosmeceutical or cosmetic products for human or veterinary use). In one preferred embodiment, said one or more bioactive agents are hydrophobic, having a water solubility of less than 10 mg/ml and a log P value greater than 1.

Although the composition of the present invention may comprise any lipophilic bioactive agent, preferably said agent is a pharmaceutical agent selected from the group consisting of anti-inflammatory drugs, calcineurin inhibitors, Janus kinase inhibitors, 5-alpha reductase inhibitors, PDE4 inhibitors, immunomodulating agents, anti-viral agents, anti-fungal agents, antibiotics, steroids, prostaglandin analogues, apoptosis inhibitors, antiparasitic agents, anaesthetic agents, statins, anti-hyperlipidemia agents, anti-hypercholesterolemia agents and anti-cancer agents.

In one preferred embodiment, the at least one bioactive agent is a 5-alpha reductase inhibitor. Particularly preferred 5-alpha reductase inhibitors include finasteride, dutasteride, and salts and derivatives of either of these compounds.

As disclosed hereinabove, the composition of the present invention may further comprise one or more solubility enhancers. Although any suitable solubility enhancer, among those well known in the art to the skilled artisan, may be used, preferably said enhancers are selected from the group consisting of polar lipids, lipids with a polar moiety such as acid, alcohol, ester, ether or amide, a cosolvent such as ethanol, diethylene glycol monoethyl ether, dimethyl isosorbide, dimethyl sulfoxide, dimethylacetamide, N-methyl-2-pyrrolidone, diethyl sebacate, dibutyl adipate, diisopropyl adipate, tocopherol, tocopherol acetate, and a lipid soluble surface active agent with a low HLB of less than about 5.0, such as sorbitan oleate or sorbitan sesquioleate.

The composition of the present invention may comprise any suitable emulsifier or combination of emulsifiers. In one preferred embodiment, however, the composition may comprise two emulsifiers, wherein one emulsifier is oil soluble, and the second emulsifier is a water-soluble surfactant.

In another aspect, the present invention is directed to a method for delivering at least one lipophilic bioactive agent into the hair follicle of a mammalian subject in need thereof, comprising the steps of: a) providing a composition as disclosed hereinabove; and b) applying said composition to the surface of the skin of said subject.

The present invention also encompasses a method for treating and/or preventing diseases and disorders of the hair follicle in a mammalian subject, comprising the steps of a) providing a composition as disclosed hereinabove, and b) applying said composition to the surface of the skin of said subject.

The method disclosed immediately hereinabove may be used to treat or prevent any disease or disorder of the hair follicle in a mammalian subject, including (but not limited to) a condition selected from the group consisting of alopecia, infectious diseases, immunological disorders, autoimmune diseases, neoplastic disorders, inflammatory conditions, and conditions in which the sebaceous glands and/or the entire hair follicle are blocked.

For the purposes of using either of the two methods disclosed hereinabove, the at least one lipophilic bioactive agent is a pharmaceutical agent selected from the group consisting of anti-inflammatory drugs, calcineurin inhibitors, Janus kinase inhibitors, 5-alpha reductase inhibitors, PDE4 inhibitors, immunomodulating agents, anti-viral agents, anti-fungal agents, antibiotics, steroids, prostaglandin analogues, apoptosis inhibitor, antiparasitic agents, anaesthetic agents, statins, anti-hyperlipidemia agents, anti-hypercholesterolemia agents and anti-cancer agents.

In some preferred embodiments, the at least one bioactive agent is finasteride and/or dutasteride. In other preferred embodiments, the bioactive agents are steroid drugs. In still other preferred embodiments, the bioactive agent is a Janus kinase inhibitor.

As explained hereinabove (and in more detail hereinbelow), the compositions of the present invention are characterized by their ability to selectively deliver the bioactive agent contained therein to the hair follicle. This results in much lower levels of the agent being detectable in either the non-follicular portions of the skin or in the blood. Thus, in one preferred embodiment of either of the methods disclosed hereinabove, following the application of the composition to the skin, the amount of the bioactive agent per unit area present in the hair follicles is higher than that the amount per unit area present in the skin of the subject being treated. Furthermore, in some embodiments of these methods, following the application of the composition to the skin, the concentration of the bioactive agent in the blood is lower than the concentration needed to achieve the desired systemic therapeutic effect. In some cases, the concentration of the bioactive agent in the blood is at least 10 times lower than the concentration that would be obtained following oral or parenteral administration of the same bioactive agent.

In some preferred embodiments of the methods of treatment of the present invention, the mammalian subject is a human subject. In other preferred embodiments, the subject is a non-human mammal.

The present invention further provides, in one aspect, a method for producing a composition as described above or a dosage form as described above, comprising the steps of dissolving the bioactive, hydrophobic, agent in the oil phase, while mixing and heating, adding the water phase with its constituents while mixing and heating and homogenizing, controlling mean droplet size, cooling the mixture and filling the final containers. Another optional method for producing a composition as described above or a dosage form as described above comprises the step of melting all ingredients except for the water phase, following which the water phase is added, after which said composition is homogenized at a selected temperature and then cooled for future use.

The methods, uses, materials, and examples that will now be described are illustrative only and are not intended to be limiting; materials, uses and methods similar or equivalent to those described herein can be used in practice or testing of the invention. Other features and advantages of the invention will be apparent from the following detailed description, and from the claims.

### BRIEF DESCRIPTION OF THE FIGURES

FIG. 1 graphically depicts the local effect of dutasteride administered orally and topically in various formulations on rate of hair growth, measured as percentage hair cover. The treatment groups (shown from left to right) are: Naïve, not treated, Oral, dutasteride suspended in a gel, FOL002 0.05% dutasteride and FOL002 0.01%

FIG. 2 is a histological photograph of the hair follicles of mouse skin following daily administration for 28 days in the alopecia testosterone model. The treatment groups: Naïve, Untreated, FOL submicron formulation with 0.05% dutasteride administered once daily, FOL submicron formulation with 0.2% dutasteride administered twice daily, FOL submicron formulation with 0.25% finasteride administered once daily. In the case of the dutasteride treatment groups, the histological sections are shown in pairs, with the treated side of the animal (late anagen/catagen) on the left, and the non-treated side (telogen) on the right.

### DETAILED DESCRIPTION OF THE INVENTION

Without being limited to any theory or mechanism, the present invention is based on the surprising finding that drugs or bioactive agents that are substantially solubilized in the internal oily phase of an emulsion and having a mean emulsion droplet size of less than about one micron, demonstrate better penetration into the hair follicles when applied on the skin with slight rubbing, while only scarcely penetrating the non-follicular portions of the skin, thereby demonstrating specific intrafollicular delivery. Furthermore, unexpectedly the mean droplet size does not change significantly either under normal shelf-life conditions or upon storage at accelerated stability conditions.

The composition disclosed herein enables targeting of drugs to the hair follicle, thus increasing the pharmacological efficacy and reducing the exposure of other organs to the drugs thereby reducing side effects, toxicity and local irritation. Moreover, the compositions are easy to apply, easily spread on the skin and are well absorbed, non-greasy, non-sticky and non-shiny.

The intrafollicular targeted composition is an emulsion comprising: a) at least one oil solvent, and b) at least one bioactive agent that is substantially solubilized in the (a) oil solvent, and c) at least one stabilizer, and d) water, and e) optionally functional excipients such as chemical stabilizers, colorant, fragrances, anti-oxidants and microbial preservatives. The droplet size of the composition is in the range of from about 200 nm to about 1,000 nm (nanometers) and the amount of (b) the bioactive agent solubilized in the (a) oil phase is sufficient to produce the desired pharmacological effect, while the amount of said bioactive agent absorbed into the skin no via the hair follicle is low and the amount absorbed systemically is also very low and is beneath the level that causes a therapeutic effect and or severe side effects or toxicity.

In one embodiment, the intrafollicular targeted composition is an emulsion comprising: a) at least on oil solvent, and b) at least one bioactive agent that is substantially solubilized in the (a) oil solvent, and c) at least one emulsifier, and d) at least one gelling agent, and e) optionally, ethanol and f) optionally a solubility enhancer and g) water, and h) optionally functional excipients such as chemical stabilizers, colorant, fragrances, antioxidants and microbial preservatives. The droplet size is in the range of from about 200 nm to about 1,000 nm (nanometers) and the amount of (b) the bioactive agent solubilized in the (a) oil phase is sufficient to produce the desired pharmacological effect, while the amount absorbed into the skin but not via the hair follicle is low and the amount absorbed systemically is also very low and is less than the level that is capable of causing systemic therapeutic effects and/or severe side effects or toxicity.

The droplet size of topical creams and lotions is not routinely measured or considered as a factor in majority of commercial drug products or cosmetic products. We have tested the mean droplet size of various marketed pharmaceutical creams and lotions and found that the average mean droplet size is from about 1 micron to about 10 microns. For example, Dermovate™ cream has mean droplet size of about 2 microns and Metrocream™ has a mean droplet size of about 3 microns. Thus, all of the tested prior art preparations are characterized inter alia by having a mean droplet size that is larger than the range of mean droplet sizes of the presently disclosed composition.

### Definitions

The term “about” as used herein refers to any value which lies within a range of ±5% of original value. For example, “about 100” refers to “95 to 105”.

As used herein, the term “mean droplet size” refers to a value which is obtained by measuring the diameters in a specific direction of droplets and dividing the sum of respective diameters of droplets by the number of measured droplets, as measured for example by a dynamic light scattering method (e.g., using a device such as the Malvern instrument DLS Nanosizer™).

As used herein, the term “Intra-follicle targeting” or “intrafollicular delivery” refers to the delivery of bioactive agents, drugs or cosmetic agents, into the vicinity of the hair follicle including the sebaceous glands. It is to be understood that this differential delivery is in favor of the hair follicle shaft over the general surface of the skin.

As used herein, the term “soluble in ethanol” means freely or at least slightly soluble in ethanol at ambient temperature. Slightly soluble is when one gram of the oil solvent is soluble in less than 100 ml of ethanol at ambient temperature.

As used herein, the term “Substantially solubilized” refer to the molecular state of the bioactive agent molecules being solubilized in the oil phase of the emulsion in an amount sufficient to exert the pharmacological effect while administered in the final product.

### Hair Follicle Diseases

Hair follicle diseases include, but are not limited to, alopecia, for example androgenic male alopecia, female alopecia, alopecia areata (AA), Chemotherapy Induced Alopecia (CIA), scarring alopecia, cicatricial alopecia; Lichen planopilaris, Frontal fibrosis alopecia, folliculitis, demodicosis, Trichodysplasia Spinulosa, pilomatrix dysplasia, atrophoderma vormicula, Rombo syndrome, Loeys-dietz syndrome, dermotrichic syndrome, keratosis folliculari, Hailey-Hailey disease, shingles, hypotrichosis, seborrhea and hirsutism. Acne of its many types such as acne vulgaris rosacea are follicular diseases that are commonly treated with various drugs such as antibiotics, benzoyl peroxide, azelaic acid and derivatives, vitamin A derivatives and disinfectants.

### The Bioactive Agent Drugs

The bioactive agent used in the present invention may be a cosmetic agent cosmeceutical, or pharmaceutical drug, for human or veterinary use, that are selected from, but not limited to: Steroidal anti-inflammatory drugs such as dexamethasone, prednisolone, methylprednisolone, monethasone, halomethasone, betamethasone, betamethasone valerate or succinate, fluorocinolone, triamcinolone, clobetasol, diflorazone, loteprednol etabonate, amcinonide and hydrocortisone and mixtures thereof; non-steroidal anti-inflammatory drugs (NSAIDs) such as, ibuprofen, diclofenac, aspirin, indomethacin, naproxen, fenoprofen, tolmetin, sulindac, meclofenamate, ketoprofen, piroxicam, tramadol, etodolac, celecoxib, nabumetone and flurbiprofen, or a salt thereof and mixtures thereof; calcineurin inhibitors such as pimecrolimus, tacrolimus and cyclosporine; JAK kinase inhibitors such as peficitinib, fedratinib, pacritinib, ruxolitinib, tofacitinib, upadacitinib, delgocotonib and baricitinib and their derivatives salts and free base as examples; PDE4 inhibitors such as crisaborole, rolipram, apremilast, roflumilast, forskolin and theophylline; 5-alpha reductase inhibitors such as dutasteride, finasteride, epristeride and; Immunosuppressing agents such as Imuran, mycophenolate mofetil; Retinoids such as acitretin, isotretinoin, tretinoin; Anti-viral agents such as ledipasvir, letermovir, docosanol (behenyl alcohol); Anti-fungal agents such as amphotericin B, glucan synthesis inhibitors such as itraconazole, caspofungin, micafungin, or anidulafungin (LY303366), econazole, terconazole, fluconazole, voriconazole, terbinafine or griseofulvin; Anti-cancer natural products including astaxanthin, lycopene, anti-oxidants, soy diadezin, genistein, polypehnols, EGCG, Coenzyme Q10, tocopherols, quercetin, resveratrol, fullerene and derivatives, azelaic acid, and its derivatives and curcumin and combinations thereof. Prostaglandins such as bimatoprost, latanoprost; Apoptosis inhibitors such as Pifithrin A; Anaesthetic drugs such as dibucaine (cinchocaine), lidocaine, benzocaine, ropivacaine, bupivacaine, etidocaine, prilocaine; Mineralocorticoid (Aldosterone) Receptor Antagonists such as spironolactone; cosmeceuticals such as vitamin A and derivatives, galbridin, glycyrrhizic acid, azelaic acid, antioxidants, betulinic acid: Antiparasitic and antiprotozoal drugs such as ivermectin, chloroquine, hydroxychloroquine, miltefosine, Crotamiton, Lindane, Disulfiram, Mesulfen, Benzyl benzoate, Spinosyn, Dapsone, Permethrin, Brimonidine; PPARγ agonists such as pioglitazone, Troglitazone, Rosiglitazone; Vasoconstrictors such as naphazoline, mephentermine; Anti-cancer drugs such as lapatinib, dasatinib, imiquimod; Anti-integrins such as lifitegrast; SP1R modulators such as siponimod, fingolimod; Anti-fibrosis drugs such as obeticholic acid; antihistamines such as destoratadine; prostaglandin analogues such as bimatoprost, latanoprost; statins and anti-hyperlipidemia and anti-hypercholesterolemia such as ezetimibe, lovastatin, simvastatin, atorvastatin.

Preferably, the bioactive agents that are included in the compositions of the present invention are lipophilic agents.

### The Oil Solvent

Oil solvents are lipidic materials that do not mix with water and are used to solubilize the bioactive agents. Oil solvents are for example triglycerides, fatty acids or alcohols and fatty esters and ethers. Triglycerides may be natural plant extracts or synthetics.

Triglycerides of plant origin include, for example, olive oil, castor oil, sunflower oil, canola oil, or peanut oils, fatty esters or acids are stearic acid, palmitic acid, oleic acid, linoleic acid, capric acid, caprylic acid, myristic acids or alcohols, and fatty acid esters and ethers such as, for example, octyl dodecanol, isopropyl myristate or any combinations thereof.

Preferred oil solvents are lipids that are soluble or miscible with ethanol, and that are liquid at room temperature and do not solidify at temperatures above 15° C., for example unsaturated triglycerides such as castor oil and olive oil, and unsaturated free fatty acid and fatty alcohols and fatty esters or ethers and various fatty acid esters and ethers such as diethyl sebacate, diisoporpyl adipate, dibutyl adipate or such as decyl oleate and octyl dodecanol.

Preferred oil solvents are lipids that are liquid at room temperature and solidify only upon refrigeration.

### The Stabilizers

Stabilizers are emulsifiers and surface-active agents that can reduce interfacial tension and reduce the tendency of phase separation and that form strong envelop around the oil droplets that avoid coalescence and aggregation, thus increase the stability.

Example of stabilizers surfactants are polysorbates and sorbitans, polyethylene glycols esters and ethers such as Myrj, Brig and sucrose esters of fatty acids or esters of polyglycerol fatty acids.

Further details of suitable dosage forms, emulsifiers, oils and stabilizers may be obtained from any standard reference work in this field, including, for example: Remington's Pharmaceutical Sciences, Mack Publishing Co, Easton, Pa., USA (1980).

### The Compositions

The compositions are made of oily solvents, stabilizers and water. The oily solvents are the oils that are proficient at solubilizing the desired concentration of the selected bioactive agent. Stabilizers are emulsifiers and gelling and suspending agents and the water phase may comprise skin humectants, such as glycerin or propylene glycol or butylene glycol or hexylene glycol and general additives such as colorant, pH buffers, fragrances and microbial preservatives and skin penetration enhancers such as diethylene glycol monoethyl ether or ethanol.

The compositions are homogenized with industry standard machinery to produce the specific mean oil droplet size of about 200 nm to about 1,000 nm and more preferably to about 400 nm to about 800 nm.

The mean droplet size does not change significantly over the product shelf life and under accelerated stability conditions.

### The Vehicle Forms

Preferred dosage forms, but not limited to, are any liquid or semi solid or solid dosage form. The topical delivery system may be a suspension, ointment, lotion, cream, foam, spray, topical patch. The vehicle may comprise any acceptable solvent and inactive ingredients as well as preservatives antioxidants and coloring agents. The delivery form may be a single dose or multiple dose form, as well known in the art of pharmaceutical, cosmetic, veterinary medicine and the art of formulation.

### The Benefits and Uses

The hair follicle targeted delivery of bioactive agents provides many benefits such as increased pharmacological efficacy, use of lower bioactive agent, sparing other organs from side effects and toxicity, reduced skin irritation, reduced blood levels and systemic side effects. Moreover, reduced side effect, toxicity and local irritation will increase patient compliance and clinical efficacy. Another benefit that supports good patient compliance is the advantageous pleasant skin feeling and usability due to fast and easy spreadability, and the non-greasy, non-tacky and non-shiny product properties of the product.

In certain preferred embodiments, the mean droplet size of the emulsion is from about 200 nm (nanometers) to about 2,000 nm. In certain preferred embodiments, the mean droplet size of the emulsion is from about 200 nm (nanometers) to about 1,500 nm. In certain preferred embodiments, the mean droplet size of the emulsion is from about 200 nm (nanometers) to about 1,200 nm. In certain preferred embodiments, the mean droplet size of the emulsion is from about 200 nm (nanometers) to about 1,000 nm. In certain preferred embodiments, the mean droplet size of the emulsion is from about 200 nm (nanometers) to about 800 nm. In certain preferred embodiments, the mean droplet size of the emulsion is from about 300 nm (nanometers) to about 700 nm. In certain preferred embodiments, the mean droplet size of the emulsion is from about 400 nm (nanometers) to about 2,000 nm. In certain preferred embodiments, the mean droplet size of the emulsion is from about 400 nm (nanometers) to about 1,200 nm. In certain preferred embodiments, the mean droplet size of the emulsion is from about 400 nm (nanometers) to about 1,000 nm. In certain preferred embodiments, the mean droplet size of the emulsion is from about 400 nm (nanometers) to about 800 nm. In certain preferred embodiments, the mean droplet size of the emulsion is from about 400 nm (nanometers) to about 700 nm. In certain preferred embodiments, the mean droplet size of the emulsion is from about 500 nm (nanometers) to about 1,200 nm. In certain preferred embodiments, the mean droplet size of the emulsion is from about 500 nm (nanometers) to about 1,000 nm. In certain preferred embodiments, the mean droplet size of the emulsion is from about 500 nm (nanometers) to about 800 nm. In certain preferred embodiments, the mean droplet size of the emulsion is from about 500 nm (nanometers) to about 700 nm.

In certain preferred embodiments the at least one oil solvent is selected from a triglyceride oil, diglyceride oil or monoglyceride oil, fatty acids and fatty alcohol or fatty esters and ethers such as: isostearic acid and derivatives, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, octyl hydroxystearate, diethyl sebacate, and mixtures thereof. Suitable liquid oil includes saturated, unsaturated or polyunsaturated oils. By way of example, the unsaturated oil may be olive oil, castor oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils or mixtures thereof, in any proportion.

In certain preferred embodiments, the oil solvent is liquid at room temperature and do not solidify upon production or storage and its melting point temperature is below 20° C., preferable below 15° C., and more preferably below 10° C. and more preferably below 5° C. and more preferably below 0° C.

In certain preferred embodiments, the oil phase of the emulsion, comprising the oil solvent and the lipophilic emulsifier and the solubility enhancer, is liquid at room temperature and do not solidify upon production or storage and its melting point temperature is below 20° C., preferable below 15° C., and more preferably below 10° C. and more preferably below 5° C. and more preferably below 0° C.

In certain embodiments, the composition comprises at least from about 1% to about 40% by weight of an oil solvent, In certain embodiments, the composition comprises at least about 4% to about 35% by weight of an oil solvent, In certain embodiments, the composition comprises at least about 6% to about 30% by weight of an oil solvent, In certain embodiments, the composition comprises at least about 8% to about 25% by weight of an oil solvent, In certain embodiments, the composition comprises at least about 10% to about 25% by weight of an oil solvent, In certain embodiments, the composition comprises at least about 12% to about 24% by weight of an oil solvent.

In certain preferred embodiments the at least one emulsifier is selected from anionic, cationic, nonionic, zwitterionic, amphoteric and ampholytic surfactants, as well as mixtures of these surfactants. Nonlimiting examples of possible surfactants include polysorbates, such as polyoxyethylene (20) sorbitan monostearate (polysorbate 60) and poly(oxyethylene) (20) sorbitan monooleate (polysorbate 80); poly(oxyethylene) (POE) fatty acid esters, such as Polyethylene Glycol 400 Monostearate (Myrj) 45, Myrj 49 and Myrj 59; poly(oxyethylene) alkylyl ethers, such as poly(oxyethylene) cetyl ether, poly(oxyethylene) palmityl ether, polyethylene oxide hexadecyl ether, polyethylene glycol cetyl ether, brij 38, brij 52, brij 56 and brij W1; sucrose esters, partial esters of sorbitol and its anhydrides, such as sorbitan monolaurate and sorbitan monolaurate; mono or diglycerides, isoceteth-20, sodium methyl cocoyl taurate, sodium methyl oleoyl taurate, sodium lauryl sulfate, triethanolamine lauryl sulfate and betaines. PEG-fatty acid esters Polyethylene glycol fatty acid diesters, Alcohol-oil transesterification derivatives of oil soluble vitamins (e.g., vitamins A, D, E, K, etc.), such as tocopheryl PEG-100 succinate (TPGS, available from Eastman), are also suitable surfactants. Further examples include Polyglycerol esters of fatty acids such as polyglyceryl 3-oleate and the polyoxyethylene-polyoxypropylene (POE-POP) block copolymers.

In one or more embodiments of the present invention, the surface-active agent is solely non-ionic, comprising one or more non-ionic surfactants. According to one or more embodiments of the present invention, two surface-active agents are selected, whereas one is water soluble or dispersible with an HLB>9 and the second is oil soluble or dispersible with HLB<9.

In one or more embodiments of the present invention, the surface-active agent comprises mono-, di- and tri-esters of sucrose with fatty acids (sucrose esters), prepared from sucrose and methyl and ethyl esters of food fatty acids or by extraction from sucroglycerides. Exemplary sucrose esters include sucrose mono-palmitate and sucrose monolaurate. Suitable sucrose esters include those having a high monoester content, which have higher HLB values.

Unlike prior art emulsion compositions, the total surfactant employed to obtain a follicle targeting emulsion that is stable, is low. Lower surfactant levels are preferred to reduce skin irritation. Total surfactant is in the range of 0.1 to 5.0% W/W of the emulsion composition, and is typically less than 3, and less than 2% W/W, or even less than 1% W/W.

In certain preferred embodiments the at least one polymer stabilizing agent is selected from naturally-occurring polymeric materials such as, locust bean gum, sodium alginate, sodium caseinate, egg albumin, gelatin agar, carrageenann gum sodium alginate, xanthan gum, quince seed extract, tragacanth gum, starch, chemically modified starches and the like, semi-synthetic polymeric materials such as cellulose ethers (e.g. hydroxyethyl cellulose, methyl cellulose, carboxymethyl cellulose, hydroxy propylmethyl cellulose), polyvinylpyrrolidone, polyvinylalcohol, guar gum, hydroxypropyl guar gum, soluble starch, cationic celluloses, cationic sugars and the like and synthetic polymeric materials such as carboxyvinyl polymers, polyvinylpyrrolidone, polyvinyl alcohol polyacrylic acid polymers, polymethacrylic acid polymers, such as Carbopol™ types or pemulene TR™, polyvinyl acetate polymers, polyvinyl chloride polymers, polyvinylidene chloride polymers and the like. Mixtures of the above compounds are also included within the scope of the present invention.

The gelling agent is present in an amount in the range of about 0.1% to about 5.0% W/W of the emulsion composition. In one or more embodiments, it is typically less than 0.5% W/W of the emulsion composition.

In certain preferred embodiments the at least one bioactive agent is water insoluble with water solubility below 10 mg/ml or below 1 mg/ml and a Log P of >1 and of from about 1.5 to about 6.0 and from about 2.0 to about 5.0 at the pH of about 3.5 to about 8.0 or at any pH within that range.

In certain preferred embodiments, the bioactive agent has a solubility of at least about 1% in the oil phase of the emulsion. In certain preferred embodiments, the bioactive agent has a solubility of at least about 2% in the oil phase of the emulsion. In certain preferred embodiments, the bioactive agent has a solubility of at least about 3% in the oil phase of the emulsion. In certain preferred embodiments, the bioactive agent has a solubility of at least about 5% in the oil phase of the emulsion.

In certain preferred embodiments, the oil phase comprises solubility enhancers for the bioactive agent. A solubility enhancer is selected as an example from polar lipids and lipids with polar moiety such as acid, alcohol, ester, ether or amide or a cosolvent such as ethanol, diethylene glycol monoethyl ether, dimethyl isosorbide, dimethyl sulfoxide, dimethylacetamide, N-methyl-2-pyrrolidone, diethyl sebacate, dibutyl adipate, diisopropyl adipate, tocopherol, tocopherol acetate, or a lipid soluble surface active agent typically with low HLB of less than about 5.0 for example, sorbitan oleate or sorbitan sesquioleate.

Partitioning of the bioactive agent between the oil phase and water phase is expected and a small fraction of the bioactive ingredient may exist in the water phase. In cases where the bioactive agent may be ionized and soluble in the water phase, the fraction that exist in the water phase may increase. However, even in such cases, efficient intrafollicular delivery is anticipated, provided that the dose of bioactive agent solubilized in the oil phase is sufficient to exert the intended pharmacological activity. The solubility of the active ingredients in the water phase and in the oil phase, may sometimes be controlled by the product pH. In such cases a pH that favors a non-ionized and less water-soluble state for the bioactive agent is used.

In certain preferred embodiments, the bioactive agent is substantially solubilized in the oil droplets, for example, with at least about 98.0% of the dose being solubilized in the oil droplet. In some preferred embodiments at least about 95.0% of the bioactive agent dose is solubilized in the oil droplets. In other preferred embodiments at least about 90.0% of the bioactive agent dose is solubilized in the oil droplets. In one preferred embodiment at least about 80.0% of the bioactive agent dose is solubilized in the oil droplets. In another preferred embodiment at least about 70.0% of the bioactive agent dose is solubilized in the oil droplets. In yet another preferred embodiment at least about 50.0% of the bioactive agent dose is solubilized in the oil droplets. In one preferred embodiment at least about 30.0% of the bioactive agent dose is solubilized in the oil droplets. In another preferred embodiment at least about 10.0% of the bioactive agent dose is solubilized in the oil droplets.

In certain preferred embodiments, the bioactive agent concentration measured in the hair follicle at from about one hour to about twelve hour and more preferably from about two hours to about six hours is at least about 1.5 times higher than the concentration measured in the skin.

In certain preferred embodiments, the bioactive agent concentration measured in the hair follicle at from about one hour to about twelve hour and more preferably from about two hours to about six hours or after repeated daily topical application, is at least about ten times higher than the concentration measured in the blood or plasma.

In certain preferred embodiments, the anticipated topical pharmacological effect is obtained while no bioactive agent is detected in the blood or plasma or the measured level of the bioactive agent in the blood or plasma is at least about ten times lower in comparison to the blood level measured after an oral dose that would elicit same topical anticipated pharmacological effect.

### Production Process

Mix the oil phase and heat to 60-65 C, add the bioactive agent, and mix until fully homogenous and dissolved, add the water phase and continue mixing and homogenizing until desired emulsion is formed. Cool to ˜25 to ˜40 C while vigorously mixing.

In certain preferred embodiments, the mean droplet size does not change significantly more than 5.0% over shelf life and/or accelerated conditions of stability. In certain preferred embodiments, the mean droplet size does not change significantly more than 10.0% over shelf life and/or accelerated conditions of stability. In certain preferred embodiments, the mean droplet size does not change significantly more than 20.0% over shelf life and/or accelerated conditions of stability. In certain preferred embodiments, the mean droplet size does not change significantly more than 30.0% over shelf life and/or accelerated conditions of stability. In certain preferred embodiments, the mean droplet size does not change significantly more than 50.0% over shelf life and/or accelerated conditions of stability.

While the invention will now be described in connection with certain preferred embodiments in the following examples so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

### EXAMPLES

### Example 1: Compositions of Dutasteride in Sub-Micron Emulsions

The general procedure of production of the compositions presented in the tables below is by dissolving the bioactive agent in the oil phase at about 60-65° C. The water phase (see for example Tables 20, 21) is heated to about 60-70° C. and added while mixing with the oil phase, followed by homogenization until desired emulsion is formed. The emulsion is cooled to ˜25 to ˜40 C while vigorously mixing.

Table 1 show formulations containing Oleic acid and IPM. The most physically stable formulations in this set were 002 and 020. Low concentrations of a gelling agent contributed to maintaining the physical stability of the formulation. In the set of formulations in which castor oil replaced the IPM (Table 2) formulations 007, 013 and 018 were most physically stable. Carbopol inhibited creaming even at very high centrifugation (10 k rpm) (formulations 004, 007, 013 and 018). Formulations with Polyglyceryl-3 oleate exhibited large droplet size and/or physical instability. A smaller droplet size is obtained with reduced concentration of oils (12% vs 24%). Table 3 shows that the reduction of oleic acid and instead addition of octyl dodecanol (total oils concentration 12%), increased droplet size. Selected formulations were tested for chemical and physical stability over time. Formulations 018, 022 and 027 exhibited physical and chemical stability for 6 months (Table 4).

TABLE 1A
Compositions with Oleic acid + Isopropyl myristate (IPM)

| | Compositions | | | | | | | | | |
|---|---|---|---|---|---|---|---|---|---|---|
| | FOLN | FOLN | FOLN | FOLN | FOLN | FOLN | FOLN | FOLN | FOLN | FOLN |
| | 002 | 009 | 011 | 017 | 003 | 008 | 012 | 020 | 024 | 028 |
| Ingredients | % w/w | % w/w | % w/w | % w/w | % w/w | % w/w | % w/w | % w/w | % w/w | % w/w |
| Isopropyl | 8.00 | 8.00 | 8.00 | 8.00 | 4.00 | 4.00 | 4.00 | 4.00 | 4.00 | 4.00 |
| myristate | | | | | | | | | | |
| Oleic acid | 16.00 | 16.00 | 16.00 | 16.00 | 8.00 | 8.00 | 8.00 | 8.00 | 8.00 | 8.00 |
| Stearyl | 0.50 | 0.50 | 0.50 | 0.50 | 0.50 | 0.50 | 0.50 | 0.50 | 0.50 | 0.50 |
| alcohol | | | | | | | | | | |
| Cetyl alcohol | 0.50 | 0.50 | 0.50 | 0.50 | 0.50 | 0.50 | 0.50 | 0.50 | 0.50 | 0.50 |
| Glycerin | 8.00 | 8.00 | 8.00 | 8.00 | 8.00 | 8.00 | 8.00 | 8.00 | 8.00 | 8.00 |
| Ethanol | 5.00 | — | 5.00 | — | 5.00 | 10.00 | — | 5.00 | 10.00 | 10.00 |
| Glyceryl | 1.00 | 1.00 | 1.00 | 1.00 | 1.00 | 1.00 | 1.00 | 1.00 | 1.00 | 1.00 |
| monostearate | | | | | | | | | | |
| Polyglyceryl-3 | — | — | — | 1.00 | 1.00 | — | — | — | 1.00 | — |
| oleate | | | | | | | | | | |
| Polysorbate | 2.00 | 2.00 | — | — | — | 2.00 | — | — | — | — |
| 20 | | | | | | | | | | |
| Sorbitan | 1.00 | 1.00 | 1.00 | — | — | 1.00 | 1.00 | 1.00 | — | 1.00 |
| oleate | | | | | | | | | | |
| Polyoxyl 40 | — | — | 2.00 | — | — | — | 2.00 | 2.00 | — | 2.00 |
| Stearate | | | | | | | | | | |
| Polysorbate | — | — | — | 2.00 | 2.00 | — | — | — | 2.00 | — |
| 60 | | | | | | | | | | |
| Xanthan gum | — | — | — | 0.15 | — | 0.15 | 0.15 | 0.15 | — | — |
| Carbopol 980 | 0.15 | 0.15 | 0.15 | — | 0.15 | — | — | — | — | 0.15 |
| Benzyl | 1.60 | 1.60 | 1.60 | 1.60 | 1.60 | 1.60 | 1.60 | 1.60 | 1.60 | 1.60 |
| alcohol | | | | | | | | | | |
| Dutasteride | 0.20 | 0.20 | 0.20 | 0.20 | 0.20 | 0.20 | 0.20 | 0.20 | 0.20 | 0.20 |
| Water to | 100.00 | 100.00 | 100.00 | 100.00 | 100.00 | 100.00 | 100.00 | 100.00 | 100.00 | 100.00 |
| Mean droplet | 657 | 1057 | 847 | 1321 | 593 | 442 | 745 | 774 | 526 | 482 |
| size (T-0) | | | | | | | | | | |
| Centrifuge at | No | Creaming | Creaming | No | Creaming | Creaming | Creaming | No | No | Creaming |
| 10K (T-0) | creaming | | | creaming | | | | creaming | creaming | |
| Mean droplet | 660 | NA | NA | NA | NA | NA | NA | NA | 602 | 597 |
| size (6 m | | | | | | | | | | |
| 25 C.) | | | | | | | | | | |
| Mean droplet | 736 | NA | NA | NA | NA | NA | NA | NA | 3248 | 1863 |
| size (6 m | | | | | | | | | | |
| 40 C.) | | | | | | | | | | |

Unexpectedly, compositions with oleic acid and IPM exhibited an inverse correlation between the concentration of ethanol and the average droplet size, i.e., with more ethanol the average droplet size was smaller and vice versa (see Table 1B).

TABLE 1B
Effect of ethanol on droplet size

| Ethanol (%) | 0 | 5 | 10 |
|---|---|---|---|
| Mean droplet | 1041 ± 235.4 | 718 nm ± 98.9 | 487 ± 34.3 |
| size (nm) | | | |
| Formulation # | 009, 012, 017 | 002, 003, 011, 020 | 008, 024, 028 |

TABLE 2
Compositions with Castor oil + Oleic acid

| | Compositions | | | | | | | | |
|---|---|---|---|---|---|---|---|---|---|
| | FOLN | FOLN | FOLN | FOLN | FOLN | FOLN | FOLN | FOLN | FOLN |
| | 006 | 007 | 014 | 015 | 018 | 004 | 013 | 021 | 022 |
| Ingredients | % w/w | % w/w | % w/w | % w/w | % w/w | % w/w | % w/w | % w/w | % w/w |
| Castor oil | 8.00 | 8.00 | 8.00 | 8.00 | 8.00 | 4.00 | 4.00 | 4.00 | 4.00 |
| Oleic acid | 16.00 | 16.00 | 16.00 | 16.00 | 16.00 | 8.00 | 8.00 | 8.00 | 8.00 |
| Stearyl alcohol | 0.50 | 0.50 | 0.50 | 0.50 | 0.50 | 0.50 | 0.50 | 0.50 | 0.50 |
| Cetyl alcohol | 0.50 | 0.50 | 0.50 | 0.50 | 0.50 | 0.50 | 0.50 | 0.50 | 0.50 |
| Glycerin | 8.00 | 8.00 | 8.00 | 8.00 | 8.00 | 8.00 | 8.00 | 8.00 | 8.00 |
| Ethanol | — | 5.00 | 5.00 | 5.00 | 10.00 | — | — | 10.00 | 10.00 |
| Glyceryl | 1.00 | 1.00 | 1.00 | 1.00 | 1.00 | 1.00 | 1.00 | 1.00 | 1.00 |
| monostearate | | | | | | | | | |
| Polyglyceryl-3 | 1.00 | — | 1.00 | — | — | — | 1.00 | — | — |
| oleate | | | | | | | | | |
| Polysorbate 20 | — | 2.00 | — | 2.00 | — | — | — | 2.00 | — |
| Sorbitan | — | 1.00 | — | 1.00 | 1.00 | 1.00 | — | 1.00 | 1.00 |
| oleate | | | | | | | | | |
| Polyoxyl 40 | — | — | — | — | 2.00 | 2.00 | — | — | 2.00 |
| Stearate | | | | | | | | | |
| Polysorbate 60 | 2.00 | — | 2.00 | — | — | — | 2.00 | — | — |
| Xanthan gum | 0.15 | — | 0.15 | 0.15 | — | — | — | 0.15 | 0.15 |
| Carbopol 980 | — | 0.15 | — | — | 0.15 | 0.15 | 0.15 | — | — |
| Benzyl alcohol | 1.60 | 1.60 | 1.60 | 1.60 | 1.60 | 1.60 | 1.60 | 1.60 | 1.60 |
| Dutasteride | 0.20 | 0.20 | 0.20 | 0.20 | 0.20 | 0.20 | 0.20 | 0.20 | 0.20 |
| Water to | 100.00 | 100.00 | 100.00 | 100.00 | 100.00 | 100.00 | 100.00 | 100.00 | 100.00 |
| Mean droplet | 1346 | 774 | 1360 | 763 | 720 | 956 | 879 | 938 | 740 |
| size (T-0) | | | | | | | | | |
| Centrifuge at | Creaming | No | Creaming | Creaming | No | No | No | Creaming | Creaming |
| 10K (T-0) | | creaming | | | creaming | creaming | creaming | | |
| Mean droplet | NA | NA | NA | NA | 805 | NA | NA | NA | 992 |
| size (6 m, 25 C.) | | | | | | | | | |
| Mean droplet | NA | NA | NA | NA | 796 | NA | NA | NA | 938 |
| size (6 m, 40 C.) | | | | | | | | | |

Compositions containing castor oil+oleic acid also exhibited an ethanol concentration dependent effect on droplet size (022 & 018 vs 004), but only in the absence of polysorbate 60+Polyglyceryl-3 oleate or polysorbate 20, which reversed this ethanol dependent effect.

TABLE 3A
Compositions based on FOLN-022 and FOLN-027

| | FOLN-022 | FOL1-002 | FOL1-007 | FOL1-004 | FOL1-005 | FOLN-027 |
|---|---|---|---|---|---|---|
| | w/w % | w/w % | w/w % | w/w % | w/w % | w/w % |
| Diethyl sebacate | — | — | — | — | 8.00 | 8.00 |
| Octyl dodecanol | — | 4.00 | | 4.00 | 4.00 | 4.00 |
| Castor oil | 4.00 | 4.00 | 4.00 | 4.00 | — | — |
| Oleic acid | 8.00 | 4.00 | 8.00 | 4.00 | — | — |
| Stearyl alcohol | 0.50 | 0.50 | 0.50 | 0.50 | 0.50 | 0.50 |
| Cetyl alcohol | 0.50 | 0.50 | 0.50 | 0.50 | 0.50 | 0.50 |
| Glycerin | 8.00 | 8.00 | 8.00 | 8.00 | 8.00 | 8.00 |
| Ethanol | 10.00 | 10.00 | 10.00 | 10.00 | 10.00 | 10.00 |
| Glyceryl monostearate | 1.00 | 1.00 | 1.00 | 1.00 | 1.00 | 1.00 |
| Sorbitan oleate | 1.00 | 1.00 | 1.00 | 1.00 | 1.00 | 1.00 |
| Polyoxyl 40 Stearate | 2.00 | 2.00 | 2.00 | 2.00 | 2.00 | 2.00 |
| Carbopol | 0.15 | 0.15 | 0.15 | 0.15 | 0.15 | 0.15 |
| Chlorocresol | — | — | 0.20 | 0.20 | 0.20 | — |
| Benzyl alcohol | 1.60 | 1.60 | — | — | — | 1.60 |
| Water to | 100 | 100 | 100 | 100 | 100 | 100 |
| Dutasteride | 0.20 | 0.20 | 0.20 | 0.20 | 0.20 | 0.20 |
| Centrifuge at 10K (T-0) | No | No | No | No | No | No |
| | creaming | creaming | creaming | creaming | creaming | creaming |
| Droplet size nM (T-0) | 657 | 1292 | 793 | 1139 | 699 | 413 |
| Mean droplet size nM (1 m, 25 C.) | 571 | 1144 | 784 | 1031 | 734 | 575 |
| Mean droplet size nM (1 m, 40 C.) | 501 | 980 | 684 | 1050 | 750 | 777 |
| API (%) T0 | 106.6 | 104.5 | 104.5 | 103.4 | 101.7 | 98.2 |
| API (%) (1 m 40 C.) | 106.6 | 106.9 | 104.5 | 101.1 | 101.1 | 104.3 |

TABLE 3B
Compositions based on FOLN-022 and FOLN-027

| | FOLN | FOLN | FOLN | FOLN | FOLN |
|---|---|---|---|---|---|
| Compositions | 022A | 022B | 027A | 027B | 027C |
| Ingredients | % W/W | % W/W | % W/W | % W/W | % W/W |
| Dutasteride | 0.5 | 0.5 | 0.5 | 0.5 | 0.5 |
| Oleic acid | 6 | 6 | — | — | — |
| Castor oil | 4 | 4 | — | — | — |
| Octyl dodecanol | — | — | 4 | 4 | 4 |
| propylene glycol | 12 | 12 | 12 | 8 | 8 |
| monocaprylate | | | | | |
| Diethyl sebacate | — | — | 8 | 8 | 8 |
| Stearyl alcohol | — | — | — | — | — |
| Cetyl alcohol | 1 | 1 | 1 | 1 | 1 |
| Glyceryl | 2 | 2 | 2 | 2 | 1 |
| monostearate | | | | | |
| Polyglyceryl-3 | — | 2 | — | — | 2 |
| oleate | | | | | |
| Sorbitan oleate | 2 | 2 | 2 | 2 | |
| Polyoxyl 40 Stearate | 2 | 2 | 2 | 3 | 3 |
| Caprylocaproyl | 3 | — | 3 | — | — |
| Polyoxyl-8 | | | | | |
| glycerides | | | | | |
| Glycerin | 6 | 6 | 6 | 6 | 6 |
| Ethanol | 10 | 10 | 10 | 10 | 10 |
| Carbopol 980 | — | — | — | — | — |
| Pemulene TR2 | 0.3 | 0.3 | 0.3 | 0.3 | 0.3 |
| Benzyl alcohol | 1.6 | 1.6 | 1.6 | 1.6 | 1.6 |
| Water to | 100 | 100 | 100 | 100 | 100 |

TABLE 4
Stability results at room temperature and accelerated condition, 25 and 40° C.,
for 6 months

| Composition | Zero time | 1M | 2M | 3M | 6M |
|---|---|---|---|---|---|
| FOLN018 dutasteride assay (%) RT | 100.5 | 104 | 101.0 | 101.4 | 101.2 |
| FOLN018 mean droplet size (nm) RT | 719.5 | 665.9 | 671.4 | 805.4 | 693.1 |
| FOLN018 dutasteride assay (%) 40oC | 100.5 | 102.5 | 99.4 | 99.2 | 96.6 |
| FOLN018 mean droplet size (nm) 40oC | 719.5 | 629.5 | 763.3 | 620.3 | 795.8 |
| FOLN022 dutasteride assay (%) RT | 100.9 | 104 | 104.0 | 101.2 | 102.7 |
| FOLN022 mean droplet size (nm) RT | 737.9 | 907.4 | 1090.8 | 937.6 | 885.8 |
| FOLN022 dutasteride assay (%) 40oC | 100.9 | 96.0 | 99.8 | 100.6 | 96.6 |
| FOLN022 mean droplet size (nm) 40oC | 737.9 | 1068.8 | 1232.6 | 864.7 | 992.4 |
| FOLN027 dutasteride assay (%) RT | 98.2 | 99.6 | 99.1 | 102.4 | 102.5 |
| FOLN027 mean droplet size (nm) RT | 412.6 | 598.7 | 449.1 | 485.9 | 574.9 |
| FOLN027 dutasteride assay (%) 40oC | 98.2 | 104.3 | 101.9 | 101.8 | 101.6 |
| FOLN027 mean droplet size (nm) 40oC | 412.6 | 776.6 | 844.5 | 1110.6 | 1396.9 |

### Example 2

### Hair Cover and Local Safety Evaluation Using Testosterone-Induced Alopecia Model in Mice

Hair on the backs of C57bl mice were shaved with electric clippers and subsequent application of ‘hair removal cream’ (Veet, Oxy Reckit Benckiser, Chartes, France). Twenty-four hours following hair removal, mice received daily subcutaneous injections of Testosterone, 1 mg/mouse, at the neck region. Twenty-four hours after the first Testosterone injection mice started receiving treatment as per Table 5 below. Topical formulations were applied onto the entire shaved back skin. The hair cover was analyzed by ImagJ software.

TABLE 5
Testosterone-induced alopecia model in mice-Hair cover (%) at Day 21

| | Testosterone | | % hair | Mean |
|---|---|---|---|---|
| | Daily | | cover at | Droplet |
| Group | injection | Treatment | 21 days | Size nm |
| naïve | No | No | 90 | — |
| Control | Yes | No | 5 | — |
| Oral finasteride | Yes | 0.02% 100 μL | 20 | NA |
| | | Once daily | | |
| Oral dutasteride | Yes | 0.01% 100 μL | 30 | NA |
| | | Once daily | | |
| Minoxidil ™ (5%) spray | Yes | 0.5% 100 μL | 25 | NA |
| | | twice daily | | |
| Topical dutasteride soluble in | Yes | 0.2% 100 μL | 30 | NA |
| ethanol/IPM/MCT/Oleic acid | | twice daily | | |
| ratio of 3/1/1/1 (solution) FOL030 | | | | |
| Topical dutasteride follicle targeting | Yes | 0.2% 100 μL | 75 | 657 |
| emulsion formulation FOLN002 | | twice daily | | |
| Topical dutasteride anhydrous | Yes | 0.2% 100 μL | 55 | 700 |
| emulsion FOLD004 | | twice daily | | |

The skin at the application site was intact with no abnormality detected after treatment with the various emulsions and Minoxidil. In contrast, skin was damaged with visual skin toxicity following treatment with dutasteride ethanol/IPM/MCT/Oleic acid solution.

The results indicate that only the topical treatment of the present invention (i.e., topical dutasteride formulation FOLN002) was successful in restoring hair cover to a significant degree (≥75%). In contradistinction, the oral finasteride and oral dutasteride treatments (administered at doses ×5 higher than the equivalent approved human dose) caused only relatively low improvements in hair cover (20 and 30%, respectively), while the anhydrous emulsions of 0.2% dutasteride (FOLD004, see Table 18 for composition) were more effective than Minoxidil or oral administration but less potent than the novel compositions (FOLN002).

### Example 3

### Hair Growth, Local Safety, DHT Expression and Blood Concentrations of Dutasteride In-Vivo

Hair on the backs of C57bl mice were shaved with electric clippers and subsequent application of ‘hair removal cream’ (Veet, Oxy Reckit Benckiser, Chartes, France). Twenty-four hours following hair removal, mice received daily subcutaneous injections of Testosterone, 1 mg/mouse, at the neck region. Twenty-four hours after the first Testosterone injection mice started receiving treatment as per Table 6 below. Topical formulations were applied onto the entire shaved back skin. The hair cover was analyzed by ImagJ software.

TABLE 6
Treatment and Group Designation

| | | | | Treatment | |
|---|---|---|---|---|---|
| Group | Test Item | n | Testosterone | Dose | Regimen |
| 1 | Naive | 3 | 0 | 0 | 0 |
| 2 | Non treated | 3 | 1 mg, SC | 0 | 0 |
| | (Negative control) | | | | |
| 3 | Dutasteride in 3% | 5 | Once daily | 10 μg/1 00 ul per | Once daily |
| | CMC gel | | | mouse, Oral | |
| 4 | Dutasteride in 3% | 5 | | 100 μl per mouse | ×28 d |
| | CMC gel | | | | |
| 5 | FOLN022 0.2% | 5 | | Topical | |
| 6 | FOLN018 0.2% | 5 | | | |
| 7 | FOLN027 0.2% | 5 | | | |
| 8 | FOLN002 0.2% | 5 | | | |
| 9 | FOLN002 0.05% | 5 | | | |
| 10 | FOLN002 0.01% | 5 | | | |
| 11 | FOLN002 0.05% | 5 | | | Twice weekly × 4 w |
| 12 | FOLN002 0.2% | 5 | | | Twice daily × 28 d |
| | (‘Internal control’) | | | | |

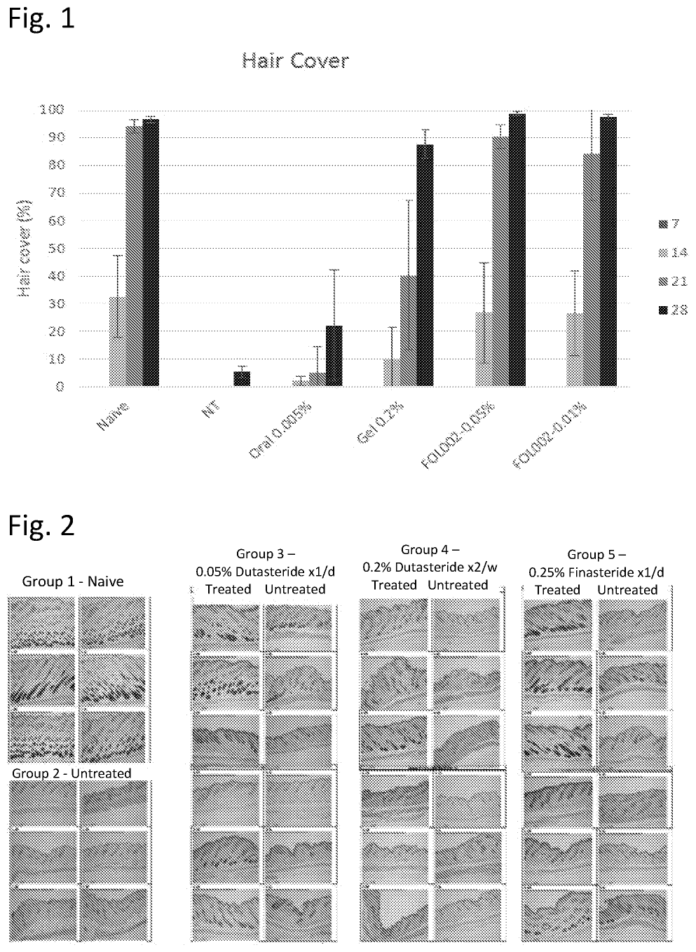

The results show that mice that were treated with as low as 0.01% dutasteride (FOLN002-0.01%) exhibited significantly improved hair cover compared to mice treated with Gel-0.2% dutasteride (Table 7). The change in hair cover caused by some of the compositions of Table 7 at the 7, 14, 21 and 28 day time points are summarized graphically in FIG. 1 (from left to right: Naïve, NT, oral 0.005%, Gel 0.2%, FOL002-0.05% and FOL002-0.01%). In addition, all formulations of the present invention, at all concentrations and regimens, reduced DHT expression in the skin. In contrast, oral administration of dutasteride (at a dose equivalent to ×5 the approved human dose) and the gel formulation did not exhibit reduced concentrations of DHT in the skin (Table 8). In sharp contrast, the blood concentrations of dutasteride were ×2.5 lower in mice treated with FOLN002-0.01% compared to the Gel-0.2% formulation that consisted ×20 more dutasteride (132 vs 325 ng/ml, respectively) (Table 9).

The effect on DHT expression in the skin was not dose dependent, suggesting that even low doses of intrafollicular dutasteride are more effective than the oral route of administration.

The highest dose administered was equivalent to ˜20 mg/kg/d. The corresponding human equivalent dose (HED) was 2 mg/kg/d, being ×200 the human oral dose. No dermal or systemic toxicity were observed following 28 d repeated once or twice daily topical applications of FOL formulations containing 0.2% dutasteride in mice.

TABLE 7
Hair cover (%) following oral and topical administration of dutasteride at
various concentrations and formulations during the 28 d study period.

| | Group Mean Hair cover (%) | | | | | SEM | | | | |
|---|---|---|---|---|---|---|---|---|---|---|
| | Study Day # | | | | | | | | | |
| | 0 | 7 | 14 | 21 | 28 | 0 | 7 | 14 | 21 | 28 |
| Naïve | 0.0 | 0.0 | 32.6 | 94.3 | 96.8 | 0 | 0 | 14.8 | 2.5 | 1.1 |
| NT | 0.0 | 0.0 | 0.0 | 0.0 | 5.6 | 0 | 0 | 0 | 0 | 2.0 |
| Oral | 0.0 | 0.0 | 2.3 | 5.2 | 22.3 | 0 | 0 | 1.7 | 9.3 | 20.1 |
| Gel-0.2% in CMC | 0.0 | 0.0 | 9.9 | 40.3 | 87.7 | 0 | 0 | 11.8 | 27.0 | 5.1 |
| FOLN022 | 0.0 | 0.0 | 18.0 | 93.8 | 99.1 | 0 | 0 | 16.7 | 1.2 | 1.0 |
| FOLN018 | 0.0 | 0.0 | 9.6 | 91.6 | 98.7 | 0 | 0 | 4.1 | 2.9 | 0.4 |
| FOLN027 | 0.0 | 0.1 | 39.0 | 94.3 | 99.5 | 0 | 0.2 | 20.5 | 2.4 | 0.8 |
| FOLN002-0.2% once daily | 0.0 | 0.0 | 28.4 | 92.4 | 98.7 | 0 | 0 | 5.6 | 3.7 | 1.0 |
| FOLN002-0.05% once daily | 0.0 | 0.3 | 26.8 | 90.5 | 98.8 | 0 | 0.6 | 18.2 | 4.4 | 0.8 |
| FOLN002-0.01% once daily | 0.0 | 0.0 | 26.4 | 84.3 | 97.5 | 0 | 0 | 15.4 | 17.0 | 1.1 |
| FOLN002 0.05% twice weekly | 0.0 | 0.0 | 6.0 | 61.3 | 95.6 | 0 | 0 | 1.6 | 21.3 | 2.5 |
| FOLN002-0.2% twice daily | 0.0 | 0.0 | 49.9 | 92.3 | 98.8 | 0 | 0 | 20.0 | 2.4 | 1.3 |

TABLE 8
Skin DHT (dihydrotestosterone) expression following oral and
topical administration of dutasteride at various concentrations and
formulations at Day 28

| | Dose | Mean DHT conc. | | P value |
|---|---|---|---|---|
| Treatment | (ug/mouse) | (nmol/L)/group | SEM | vs NT |
| NO treatment (NT) | 0 | 23.61 | 0.13 | |
| Dutasteride (oral) | 5-10 × 1/d | 22.91 | 0.02 | ns |
| Dutasteride 0.2% in | 200 × 1/d | 22.48 | 0.02 | ns |
| CMC gel | | | | |
| FOLN022 0.2% | 200 × 1/d | 21.97 | 0.77 | <0.05 |
| FOLN018 0.2% | 200 × 1/d | 17.44 | 1.66 | <0.0005 |
| FOLN027 0.2% | 200 × 1/d | 18.33 | 0.65 | <0.00001 |
| FOLN002 0.2% | 200 × 1/d | 20.81 | 0.86 | <0.005 |
| FOLN002 0.05% | 50 × 1/d | 19.89 | 1.02 | <0.005 |
| FOLN002 0.01% | 10 × 1/d | 19.31 | 1.92 | <0.01 |
| FOLN002 0.05% | 50 × 2/week | 18.62 | 1.83 | <0.005 |
| FOLN002 0.2% | 200 × 2/d | 18.58 | 0.68 | <0.0005 |

TABLE 9
Blood concentration of dutasteride following oral and topical administration
of dutasteride at various concentrations and formulations at Day 28

| Formulation | Suspension | GEL-0.2% | FOLN002-0.05% | FOLN002-0.01 % |
|---|---|---|---|---|
| Route | Oral | Topical | Topical | Topical |
| Daily dose (μg/mouse) | 5-10 | 200 | 50 | 10 |
| Blood cone (ng/ml) | 39 | 325 | 394 | 132 |

### Example 4

### Topical Effect on Hair Cover Using the Testosterone-Induced Alopecia Model in Mice

Induction of Alopecia: Hair on the backs of C57bl mice were shaved with electric clippers and subsequent application of ‘hair removal cream’ (Veet, Oxy Reckit Benckiser, Chartes, France). Twenty-four hours following hair removal, mice received daily subcutaneous injections of Testosterone, 1 mg/mouse, at the neck region. Twenty-four hours after the first Testosterone injection mice started receiving treatment as per Table 10 below.

Treatment was applied to a small, designated area of 2.5×1 cm on the right side of the back, along the spine. Repeated applications to the same site were done by using a stencil of the specified size. The rest of the back skin was kept untreated. The hair cover at the designated area of 2.5×1 cm treated and non-treated skin was analyzed by ImagJ software.

The results as shown in Table 11 clearly demonstrated, once again, that the effect of FOL formulations was mainly topical as the hair cover at the treated site was significantly higher compared to the non-treated site. The histological evaluation of treated and non-treated skin showed treatment related effect on hair growth cycle and follicle development state, which correlate with the clinically observed hair cover. The right skin sites treated with topical dutasteride or finasteride exhibited enhanced hair growth in comparison to non-treated mice, with significant anagen and catagen state over the resting telogen state of the hair follicles, respectively. These histological results are shown in FIG. 2. No dermal toxicity was observed.

TABLE 10
Treatment and Group Designation

| | | | | Treatment | | |
|---|---|---|---|---|---|---|
| Group | Test Item | n | Testosterone | Dose | Regimen | Euthanasia |
| G1 | Naïve | 3 | 0 | 0 | 0 | Day 18 |
| G2 | Non-Treated Controls | 3 | 1 mg/100 ml | 0 | 0 | Day 18 |
| G3 | FOL027-0.05% | 3 | SC ×1/d | 25 ml per | Once Daily ×11 | Day 12 |
| | | 3 | | mouse | Once Daily ×14 | Day 15 |
| | | 3 | | Topical | Once Daily ×17 | Day 18 |
| G4 | FOL027-0.2% | 3 | | | Twice weekly ×4 | Day 12 |
| | | 3 | | | Twice weekly ×5 | Day 15 |
| | | 3 | | | Twice weekly ×6 | Day 18 |
| G5 | Finasteride 0.25% in | 3 | | | Once Daily ×11 | Day 12 |
| | FOL027 | 3 | | | Once Daily ×14 | Day 15 |
| | | 3 | | | Once Daily ×17 | Day 18 |

TABLE 11
Hair cover (%) at treated vs non treated sites on back skin of mice

| | Day-1 | | Day 8 | | Day 12 | | Day 15 | | Day 18 | |
|---|---|---|---|---|---|---|---|---|---|---|
| MEAN | Right | Left | Right | Left | Right | Left | Right | Left | Right | Left |
| Non treated (NT) | 0 | 0 | 0 | 0 | 0 | 0 | 0 | 0 | 0 | 1 |
| FOL027-0.05% | 0 | 0 | 0 | 0 | 0 | 0 | 16 | 1 | 34 | 3 |
| Once Daily | | | | | | | | | | |
| Dutasteride | | | | | | | | | | |
| FOL027-0.2% | 0 | 0 | 0 | 0 | 1 | 0 | 9 | 2 | 10 | 1 |
| Twice Weekly | | | | | | | | | | |
| Dutasteride | | | | | | | | | | |
| FOL027-0.25% | 0 | 0 | 1 | 0 | 0 | 0 | 28 | 1 | 46 | 1 |
| Once Daily | | | | | | | | | | |
| Finasteride | | | | | | | | | | |
| SEM | | | | | | | | | | |
| Non treated | 0 | 0 | 0 | 0 | 0 | 0 | 0 | 0 | 0 | 1 |
| Duta 0.05% ×1/d | 0 | 0 | 0 | 0 | 0 | 0 | 5 | 1 | 14 | 2 |
| Duta 0.2% ×2/w | 0 | 0 | 0 | 0 | 0 | 0 | 4 | 1 | 7 | 1 |
| Fina 0.25% ×1/d | 0 | 0 | 0 | 0 | 0 | 0 | 15 | 1 | 20 | 1 |

### Example 5

### In-Vitro Transdermal Delivery of Dutasteride

Penetration of dutasteride, through full thickness pig ear skin during 28 h was evaluated using the Franz Cell TDD system. Four FOL formulations containing 0.2% dutasteride were tested in quadruplicates. Dutasteride analysis was conducted using HPLC. No Dutasteride was detected in the receiver fluid.

### Example 6

### PK and Safety of Topical Dutasteride in Pigs

The local safety and systemic exposure following 28 d repeated topical application of FOLN027, at 2 concentrations was tested in 20 kg domestic male pigs. Formulations were applied topically once daily for a period of 28 consecutive days as per Table 12 below. Clinical observations and Draize scoring were done daily. Biopsies for histopathology and blood for bioanalysis of dutasteride were collected throughout the study at 3 pre-determined time points. The dutasteride concentrations measured in plasma are summarized in Table 13. These results show that a significant blood level of dutasteride is measurable only with the highest topical dose used (6.3 mg/day). Even in this case, the plasma concentration was only about half that seen in the case of oral treatment.

TABLE 12
Treatment and group designation

| | | | Once daily topical application ×28 Days | | | |
|---|---|---|---|---|---|---|
| | | | | | | Dose of |
| | Body | | | Volume of Administration | | Dutasteride |
| Group | Weight | n | Formulation | Right | Left | (mg/d) |
| 1 | ~20 kg | 33 | FOLN027 0.05% | 4% BSA; 1 ml | — | 0.5 |
| 2 | ~20 kg | 33 | FOLN027 0.2% | 4% BSA 12 × 20 cm; 1 ml | — | 2 |
| 3 | ~20 kg | 33 | FOLN027 0.2% | 12% BSA 18 × 40 cm; 3 ml | — | 6.3 |

TABLE 13
Plasma concentrations of dutasteride (ng/ml)

| | | Plasma Concentration of Dutasteride (ng/ml) | | | |
|---|---|---|---|---|---|
| | | Study Day # | | | |
| Group | Dose | 0 | 9 | 18 | 29 |
| 1 | 0.5 mg/d | 0.39 | 0.58 | 0 | 0.36 |
| 2 | 2 mg/d | 0.39 | 0.98 | 0.22 | 0.04 |
| 3 | 6.3 mg/d | 0.47 | 3.21 | 18.23 | 21.44 |

| | | |
|---|---|---|
| Human* | 0.5 mg PO | 23.60 |

*From the literature

There was no systemic exposure to Dutasteride following 28 d repeated topical application of FOLN027 onto 4% body surface area (BSA), of up to 2 mg/d, in young pigs weighing ˜20 kg. The evaluation of the local safety of dutasteride FOL formulations following 28 consecutive days of topical application in pigs showed no toxic effects.

### Example 7

### Skin Follicle Targeting of Dutasteride Emulsion in a Human Subject.

Nine square cm (3 cm×3 cm) squares were marked on forearm. Samples of 100 microliters of various 0.5% Dutasteride formulations were applied and rubbed in for 30 second. Three hours afterward, each square was tape stripped for 5 times followed by washing with plenty of soap and water. Each square was treated with hot wax and all hair including about 80 to about 120 hair follicles obtained per sample were stripped of and collected. The amount of Dutasteride in the hair follicles was measured by UHPLC. Formulation FOL040 show 0.67 mcg/cm2 and FOL041 0.71 mcg/cm2 tested 3 hours after single application, while same dose of dutasteride solubilized in solvent show much lower amount of 0.29 mcg/cm2. In another test the samples test items were applied twice a day for two days and in the morning of the third day, followed by hot wax stripping and hair follicle collection three hours or six hours after the final application, Formulation 040 show 2.9 mcg/cm2 after three hours and 1.3 mcg/cm2 after six hours and formulation 041 showed 3.1 mcg/cm2 after three hours and 1.3 mcg/cm2 after six hours. Two other formulations of same dose of dutasteride 1) solubilized in solvents, ethanol/IPM/MCT/Oleic acid ratio of 3/1/1/1 forming a clear solution, and 2) formulated in oil-in-water emulsion FOL042, with mean droplet size of about 5 microns that was produced of the same composition as FOL041 but without high shear homogenization, showed 1.4 mcg/cm2 after 3 hours and 1.0 mcg/cm2 after six hours and 0.4 mcg/cm2 and 0.2 mcg/cm2 respectively. Droplet size of formulation FOL040 was 811 nm, formulation FOL041 was 480 nm and formulation FOL042 5,600 nm. These results indicate that the two compositions of the present invention showed significant accumulation within the hair follicle and that smaller mean droplet size performed better in follicular targeting of the drug.

TABLE 14
Formulations FOL040, FOL041 and FOL042

| | Composition | | Composition | |
|---|---|---|---|---|
| | FOL040 | | FOL041 | FOL042 |
| Ingredient | % W/W | Ingredient | % W/W | % W/W |
| Dutasteride | 0.5 | Dutasteride | 0.5 | 0.5 |
| Castor oil | 14 | Medium-chain | 10 | 10 |
| | | triglycerides | | |
| Isopropyl Myristate | 11 | Polysorbate 80 | 2 | 2 |
| Cetyl alcohol | 4 | Glyceryl | 1 | 1 |
| | | monostearate | | |
| Sorbitan stearate | 3 | Phenoxyethanol | 2 | 2 |
| Polysorbate 60 | 3 | Water | 84.998 | 84.998 |
| Carbomer 980p | 0.3 | Total | 100 | 100 |
| Water | 36.6 | | | |
| Propylene glycol | 13 | | | |
| Polyethylene | 13 | | | |
| glycol 200 | | | | |
| Benzyl alcohol | 1.6 | | | |
| Total | 100 | | | |
| Mean droplet size, | 811 | | 480 | 5,600 |
| nm | | | | |

### Example 8

### Compositions of Various Drugs Solubilized in the Oil Internal Phase of in Sub-Micron Emulsions.

TABLE 15
Composition of various drug substances in sub-micron emulsions

| | Compositions | | | | |
|---|---|---|---|---|---|
| Ingredients and | CYC | RUX | TRM | FLT | TER |
| LogP | % W/W | % W/W | % W/W | % W/W | % W/W |
| Cyclosporin LogP | 0.20 | — | — | — | — |
| 3.64 | | | | | |
| Ruxolitinib logP, 2.48 | — | 0.20 | — | — | — |
| Triamcinolone | — | — | 0.1 | — | — |
| acetonide 2.53 | | | | | |
| (LogP) | | | | | |
| Flutamide | — | — | — | 0.50 | — |
| logP 3.35 | | | | | |
| Terbinafine | — | — | — | — | 1.00 |
| logP 5.53 | | | | | |
| Capric/Caprylic | — | — | — | — | — |
| triglyceride | | | | | |
| Sesame oil | — | — | — | 4.00 | — |
| Diethyl sebacate | — | 16.00 | — | — | — |
| Octyl dodecanol | — | 8.00 | — | — | — |
| Isopropyl myristate | — | — | 4.00 | — | — |
| Castor oil | 8.00 | — | — | — | 4.00 |
| Oleic acid | 16.00 | — | 8.00 | 8.00 | 8.00 |
| Stearyl alcohol | 0.50 | 0.50 | 0.50 | 0.50 | 0.50 |
| Cetyl alcohol | 0.50 | 0.50 | 0.50 | 0.50 | 0.50 |
| Glycerin | 8.00 | 8.00 | 8.00 | 8.00 | 8.00 |
| Ethanol | 5.00 | 10.00 | 10.00 | 10.00 | 10.00 |
| Glyceryl | 1.00 | 1.00 | 1.00 | 1.00 | 1.00 |
| monostearate | | | | | |
| Polyglyceryl-3 oleate | — | — | — | — | — |
| Polysorbate 20 | 2.00 | — | 2.00 | — | 2.00 |
| Sorbitan oleate | 1.00 | 1.00 | 1.00 | 1.00 | 1.00 |
| Polyoxyl 40 Stearate | — | 2.00 | — | 2.00 | — |
| Polysorbate 60 | — | — | — | — | — |
| Xanthan gum | — | — | 0.15 | 0.15 | 0.15 |
| Carbopol 980 | 0.15 | 0.15 | — | — | — |
| Benzyl alcohol | 1.60 | 1.60 | 1.60 | 1.60 | 1.60 |
| Dutasteride | — | — | — | — | — |
| Water to | 100.00 | 100.00 | 100.00 | 100.00 | 100.00 |

TABLE 16
Composition of various drug substances in sub-micron emulsions

| | Compositions | | | | | |
|---|---|---|---|---|---|---|
| | BZY | BZY | TAZ | TAZ | ROF | PAC |
| Ingredients | % w/w | % w/w | % w/w | % w/w | % w/w | % w/w |
| Benzoyl peroxide | 1.00 | 1.00 | — | — | — | — |
| LogP 2.75 | | | | | | |
| Tazarotene | — | — | 0.10 | 0.10 | — | — |
| logP 5.6 | | | | | | |
| Roflumilast | — | — | — | — | 0.20 | — |
| logP 4.47 | | | | | | |
| Pacritinib | — | — | — | — | — | 0.40 |
| logP 4.58 | | | | | | |
| Tocopherol | — | — | — | — | — | 0.2 |
| acetate LogP 10.8 | | | | | | |
| Diethyl sebacate | — | — | — | — | — | 4.00 |
| Capric/Caprylic | — | 4.00 | 4.00 | 4.00 | — | — |
| triglyceride | | | | | | |
| Isopropyl myristate | 4.00 | — | 4.00 | — | — | — |
| Castor oil | — | — | — | — | 4.00 | 4.00 |
| Oleic acid | 8.00 | 8.00 | 4.00 | 8.00 | 8.00 | 4.00 |
| Cetostearyl | 1.00 | 1.00 | 1.00 | 1.00 | 1.00 | 2.00 |
| alcohol | | | | | | |
| Glycerin | 8.00 | 8.00 | 8.00 | 8.00 | 8.00 | 8.00 |
| Ethanol | — | — | 10.00 | 10.00 | 10.00 | 10.00 |
| Glyceryl | 1.00 | 1.00 | 1.00 | 1.00 | 1.00 | 1.00 |
| monostearate | | | | | | |
| Polysorbate 20 | — | — | 2.00 | — | — | — |
| Sorbitan oleate | 1.00 | 1.00 | 1.00 | 1.00 | 1.00 | 1.00 |
| Polyoxyl 40 | 2.00 | 2.00 | — | 2.00 | 2.00 | 2.00 |
| Stearate | | | | | | |
| Carbopol 980 | 0.15 | 0.15 | 0.15 | 0.15 | 0.15 | 0.15 |
| Benzyl alcohol | 1.60 | 1.60 | 1.60 | 1.60 | 1.60 | 1.60 |
| Water to | 100.00 | 100.00 | 100.00 | 100.00 | 100.00 | 100.00 |

### Example 9

### Pacritinib in Pig's Ears Follicle

Pacritinib was formulated either in an oil-in-water emulsion with mean droplet size of about 650 nm (present invention), a similar emulsion with a mean droplet size of 3 microns (out of scope of present invention) or in an ethanol and oil solution (prior art composition). Each of these compositions was applied, separately, on freshly obtained pig's ears and rubbed in for 30 seconds. One hour after the application, the skin was stripped five times with sellotape, and hair follicle were collected using wax stripping. The amount of Pacritinib in the hair follicle samples was measured by UHPLC. The results obtained indicate that significantly higher follicular levels of the Janus kinase inhibitor Pacricitnib were seen following treatment with a composition of the present invention, as compared with treatment with the prior art compositions.

TABLE 17
Results of Pacritinib amount in hair follicles

| | Pig's ears follicles |
|---|---|
| Treatment | amount mcg/cm2 |
| None | Below limit of |
| | quantification |
| Solution: ethanol/IPM/MCT/Oleic acid ratio of | 0.42 |
| 3/1/1/1 | |
| Intra follicular emulsion formulation PAC with | 0.85 |
| mean droplet size of 650 nm | |
| Emulsion formulation PAC (without high shear | Below limit of |
| homogenization) with mean droplet size of | quantification |
| 3 microns | |
| (D90 1 to 10 microns) | |

### Example 10

### Comparative Skin Feeling of Placebo Sub-Micron Emulsions and Anhydrous Emulsions

Formulations FOLD001, 002, 003 and 004 of Table 18 and formulations FOLN002, 003, 004, 006, 007, 008, 011, 012, 013, 014, 016, 017, 018, 020, 021, 022 and 024 of Tables 1 and 2, were blindly tested on twelve healthy volunteers ages 30 to 65. The subjects filled in a questionnaire for general skin feeling, spreadability, absorption, greasiness, tackiness, and shine. The formulations were also tested for sprayability out of a spray pump. Selected formulations FOLN002 (table 1) and FOLD004 (Table 18) were also tested on male alopecia bald persons.

Formulation FOLD001 to 004 were of poor or low skin feeling, poor to medium spreadability, greasy tacky and shiny, with moderate absorption and low to medium general score. They were also non-sprayable. All FOLN formulations from tables 1 and 2 were significantly superior, with good general score, good spreadability and good absorption. They were non-greasy, non-tacky, non-shiny and sprayable. FOLN002 was superior on head skin feeling over FOLD004 that had greasy skin feeling, and inferior in spreadability and absorption.

TABLE 18
Low water, oil-in-polyol emulsions placebo formulations

| | Formulations | | | |
|---|---|---|---|---|
| | FOLD001 | FOLD002 | FOLD003 | FOLD004 |
| Composition | % W/W | % W/W | % W/W | % W/W |
| Capric/caprylic | 8.00 | — | — | — |
| triglycerides | | | | |
| Octyl dodecanol | 8.00 | — | 5.00 | — |
| Castor oil | — | — | — | 6.00 |
| Oleic acid | — | 15.00 | 10.00 | 8.00 |
| Stearyl alcohol | — | — | — | 2.00 |
| Cetyl alcohol | 2.00 | 1.00 | 2.10 | — |
| Glycerin | 50.00 | 10.00 | — | — |
| Propylene glycol | — | 40.00 | 32.00 | 31.00 |
| Polyethylene glycol 200 | 11.00 | 15.00 | 30.00 | 30.00 |
| Glyceryl monostearate | 2.00 | 1.00 | 2.00 | 2.00 |
| Sorbitan oleate | — | 1.00 | — | — |
| Polyoxyl 40 Stearate | 2.20 | — | 2.00 | 2.00 |
| Benzyl alcohol | 1.60 | 1.60 | 1.60 | 1.60 |
| Water | 15.20 | 15.40 | 15.30 | 17.40 |
| Total | 100.00 | 100.00 | 100.00 | 100.00 |

### Example 11

### The Ratio Between Follicle and Skin Concentrations of Progesterone

Formulations with progesterone were prepared as per Table 19 below. Formulations were applied on to 3×3 cm pig's ear skin followed by massage for 30 seconds. Skin samples were incubated at 37° C. Following incubation, the skin surface was thoroughly cleansed. Follicles and adjacent treated skin (without follicles) were collected using biopsy punch. Progesterone was extracted in methanol and subsequently extracts were subjected to ELISA for the determination of progesterone.

TABLE 19
Effect of emulsification API solubilization on specific intrafollicular delivery of Progesterone

| Progesterone | | | | NON solubilized | | Suspended | |
|---|---|---|---|---|---|---|---|
| in sub-micron | | | | progesterone | | progesteron | |
| emulsion lotion | FOL03-027 | Progesterone in solution | FOL03-030 | sub-micron emulsions | FOL3-010 | in a gel | FOL3-011 |
| Ingredient name | % w/w | Ingredient Name | % w/w | Ingredient name | % w/w | Ingredient name | % w/w |
| Diethyl sebacate | 8 | Medium-Chain Triglyceride | 20 | Mineral oil | 8 | Water | 98 |
| Octyl dodecanol | 4 | Isopropyl myristate | 20 | Vaselin | 4 | CMC | 0.5 |
| Glyceryl monostearate | 1 | Ethanol | 40 | Cetyl alcohol | 0.5 | Benzyl alcohol | 1 |
| Polyoxyl 40 stearate | 2 | Oleic acid | 19.5 | Glycerin | 8 | Progesterone | 0.5 |
| Sorbitan oleate | 1 | Dutasteride | 0.5 | Glyceryl monostearate | 1 | Total | 100 |
| Stearyl alcohol | 0.5 | Total | 100 | Sorbitan oleate | 1 | Follicle to skin | 1.2 |
| Cetyl alcohol | 0.5 | Follicle to skin | 1.5 | Polyoxyl 40 Stearate | 2 | content ration | |
| Water | 62.55 | content ration | | Carbopol 980 | 0.15 | | |
| Carbomer | 0.15 | | | Benzyl alcohol | 1.6 | | |
| Carrageenan-lambda | 0.5 | | | Water | 73.25 | | |
| Ethanol | 10 | | | Progesterone | 0.5 | | |
| Glycerin | 8 | | | Total | 100 | | |
| Benzyl alcohol | 1.6 | | | Follicle to skin | 1.4 | | |
| Progesterone | 0.5 | | | content ration | | | |
| Total | 100 | | | | | | |
| Follicle to skin | 2 | | | | | | |
| content ration | | | | | | | |

Progesterone formulated in composition 027, a sub-micron lotion where the progesterone was fully solubilized in the oil phase of the emulsion, show the highest ratio of follicle to skin content; a higher ratio than that obtained following the application of progesterone solubilized in solution (030), a higher ratio than that obtained following the application of sub-micron emulsion with oils that do not solubilize the progesterone (010) and a higher ratio than that obtained following the application of progesterone powder suspended in 0.3% CMC gel in water (011). The results show higher intrafollicular delivery of a hydrophobic agent (progesterone) formulated in accordance with the present invention.

### Example 12

### Effect of Droplet Size on Specific Intrafollicular Delivery of Dutasteride

Pig ears were gently cleansed with water and dried with a gauze pad and incubated for 30 min at 32° C. in humidified oven prior to Test Formulation application. The application sites, 3×3 cm, were marked using a permanent marker. Each formulation, 50 μL, was applied onto the skin in quadruplicates. Formulation FOLN002 was massaged into the skin for 30″ followed by 3h incubation at 32° C. in a humidified oven. The skin was cleaned using a dry gauze pad followed by tape striping 2 strips ×5 times.

Hair follicles from each application site were collected by wax striping. Dutasteride was extracted from the wax and skin samples by incubation with agitation in methanol at 50° C. for 48 h. Thereafter, dutasteride was quantified by UPLC. Table 20 presents the mean ratio of dutasteride found in follicles vs. skin. The formulation with the smaller droplet size (<1 mM) delivered significantly more dutasteride into the follicle as compared to skin (×5), whereas similar amounts of dutasteride were found in the hair follicles and skin when droplet size was >1 mM. The results show higher intrafollicular delivery of a hydrophobic agent (dutasteride) formulated in accordance with the present invention.

TABLE 20
Specific intrafollicular delivery of dutasteride into hair follicle (HF)

| FOLN002 | Droplet size (nM) | Mean ± SD HF/skin ratio | Significance |
|---|---|---|---|
| A | 2456 | 1.6 ± 0.68 | |
| B | 716 | 5.0 ± 0.62 | p = 0.05 |

### Example 13

### Intrafollicular Delivery of Various Drugs Solubilized in Oil Phase of Sub-Micron Emulsions

Tacrolimus 5.35%, cyclosporine 5.45%, 10% lovastatin 5%, Ezetimibe 5.1% and crisaborole 5% were dissolved in the oil phase as per Table 21 and azelaic acid 7.5% was dissolved in the oil phase as per Table 24 below at 65° C. Water phase heated to about 65° C. was added with vigorous mixing and immediately homogenized with a high shear homogenizer for 1 min followed by addition of the ethanol to the emulsion and homogenized for an additional 1 minute and cooling to ambient temperature. The physical stability of the formulations was tested after at least 1 week at 5, 25 and 40° C. (Table 23).

The extent of specific intrafollicular delivery and or biological activity are evaluated using specific ELISA kits as per the method described above in Example 11.

TABLE 21
Prototype Formulation

| Phase | Category | Ingredient Name | % w/w |
|---|---|---|---|
| Oil phase | Oil | Diethyl sebacate | 8 |
| | Oil | Octyl dodecanol | 4 |
| | Emulsifier | Glyceryl monostearate | 1 |
| | Emulsifier | Polyoxyl 40 stearate | 2 |
| | Emulsifier | Sorbitan oleate | 1 |
| | Fatty acid alcohol | Stearyl alcohol | 0.5 |
| | Fatty acid alcohol | Cetyl alcohol | 0.5 |
| | Preservative | Benzyl alcohol | 1.6 |
| Water phase | Gelling agent | Carbomer | 0.15 |
| | | Glycerin | 8 |
| | | Water to | 100 |
| EtOH | | Ethanol | 10 |

TABLE 22
Physical properties at T-0

| | Tacrolimus | Cyclosporine | Lovastatin | Ezetimibe | Crisaborole |
|---|---|---|---|---|---|
| | (5.35%) | (5.45%) | (5%) | (5.1%) | (5%) |
| pH | 5.42 | 5.39 | 5.36 | 5.55 | 5.59 |
| DLS | 676 nm | 781 nm | 540 nm | 390 nm | 488 nm |
| Odor | No odor | No odor | No odor | No odor | No odor |
| Color | White | White | White | White | White |
| Texture | Lotion | Cream | Lotion | Cream | Cream |
| Centrifugation | Stable | Stable | Stable | Stable | Stable |
| 3 k | Stable | Stable | Stable | Stable | Stable |
| 5 k | | | | | |
| Microscopic | No crystals, | No crystals, | No crystals, | No crystals, | No crystals, |
| observation | homogenic | homogenic | homogenic | homogenic | homogenic |
| | emulsion | emulsion | emulsion | emulsion | emulsion |

TABLE 23
Physical properties after 1 week at 5, 25 and 40° C.

| | Tacrolimus | Cyclosporine | Lovastatin | Ezetimibe |
|---|---|---|---|---|
| | (5.35%) | (5.45%) | (5.0%) | (5.1%) |
| DLS | 673 nm | 1004 nm | 404 | 471 |
| | (25° C.) | (25° C.) | (25° C.) | (25° C.) |
| Odor | No odor | No odor | No odor | No odor |
| Color | White | White | White | White |
| Texture | Cream | Cream | Cream | Cream |
| Centrifugation 3 k | Stable | Stable | Stable | Stable |
| 5 k | Stable | Stable | Stable | Stable |
| Microscopic | No crystals, | No crystals, | No crystals, | No crystals, |
| observation | homogenic | homogenic | homogenic | homogenic |
| | emulsion | emulsion | emulsion | emulsion |

TABLE 24
Composition with azelaic acid

| Phase | Category | Ingredient Name | % w/w |
|---|---|---|---|
| Oil phase | Oil | Oleic acid | 16 |
| | Oil | Castor oil | 8 |
| | Emulsifier | Glyceryl monostearate | 1 |
| | Emulsifier | Polyoxyl 40 stearate | 2 |
| | Emulsifier | Sorbitan oleate | 1 |
| | Fatty acid alcohol | Stearyl alcohol | 0.5 |
| | Fatty acid alcohol | Cetyl alcohol | 0.5 |
| | Cosmeceutical | Azelaic acid | 2.25 |
| | Preservative | Benzyl alcohol | 1.0 |
| Water phase | Gelling agent | Carbomer | 0.35 |
| | | Glycerin | 8 |
| | | Water to | 100 |
| EtOH | | Ethanol | 10 |

Physical properties

| | |
|---|---|
| Texture | Lotion |
| DLS | 1071 nm |
| Odor | No odor |
| Centrifugation 3 k | Stable |
| 5 k | Stable |
| Color | White |
| pH | 4.9 |
| Microscopic | No crystals, |
| observation | homogenic emulsion |

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive.

1. A composition in the form of an oil-in-water emulsion, comprising one or more lipophilic bioactive agents, one or more oil solvents, one or more emulsifiers and water;
wherein said one or more bioactive agent is substantially dissolved in the internal oil phase of said emulsion;
wherein the mean droplet size of said emulsion is in the range of about 200 to about 1000 nm;
wherein the one or more oil solvents is soluble or miscible with ethanol; wherein said one or more oil solvents are selected from the group consisting of a natural or synthetic unsaturated triglycerides, fatty acids, fatty alcohols, fatty esters or fatty ethers thereof;
and wherein said one or more oil solvents have a melting point of less than about 15° C.

2. The composition according to claim 1, wherein said composition further comprises at least one solubility enhancer.

3. The composition according to claim 1, wherein said composition further comprises ethanol.

4. The composition according to claim 1, wherein the droplet size of the oil-in-water emulsion is in the range of about 400 to about 800 nm.

5. The composition according to claim 1, wherein the one or more bioactive agents is hydrophobic having a water solubility <10 mg/ml and log P>1.

6. The composition according to claim 1 wherein the one or more bioactive agents is a cosmetic agent or pharmaceutic agent suitable for human or veterinary use.

7. The composition according to claim 6, wherein the at least one bioactive agent is a pharmaceutical agent selected from the group consisting of anti-inflammatory drugs, calcineurin inhibitors, Janus kinase inhibitors, 5-alpha reductase inhibitors, PDE4 inhibitors, immunomodulating agents, anti-viral agents, anti-fungal agents, antibiotics, steroids, prostaglandin analogues, apoptosis inhibitors, antiparasitic agents, anaesthetic agents, statins, anti-hyperlipidemia agents, anti-hypercholesterolemia agents and anti-cancer agents.

8. The composition according to claim 7, wherein the at least one bioactive agent is a 5-alpha reductase inhibitor, selected from finasteride, dutasteride, their salts or derivatives.

9. The composition according to claim 2, wherein the at least one solubility enhancer is selected from the group consisting of polar lipids, lipids with a polar moiety such as acid, alcohol, ester, ether or amide, a cosolvent such as ethanol, diethylene glycol monoethyl ether, dimethyl isosorbide, dimethyl sulfoxide, dimethylacetamide, N-methyl-2-pyrrolidone, diethyl sebacate, dibutyl adipate, diisopropyl adipate, tocopherol, tocopherol acetate, and a lipid soluble surface active agent with a low HLB of less than about 5.0, such as sorbitan oleate or sorbitan sesquioleate.

10. The composition according to claim 1, comprising two emulsifiers, wherein one emulsifier is oil soluble, and the second emulsifier is a water-soluble surfactant.

11. A method for delivering at least one lipophilic bioactive agent into the hair follicle of a mammalian subject in need thereof, comprising the steps of: a) providing a composition according to claim 1; and b) applying said composition to the surface of the skin of said subject.

12. A method for treating and/or preventing diseases and disorders of the hair follicle in a mammalian subject, comprising the steps of a) providing a composition according to claim 1, and b) applying said composition to the surface of the skin of said subject.

13. The method according to claim 12, wherein the disease or disorder of the hair follicle is selected from the group consisting of alopecia, infectious diseases, immunological disorders, autoimmune diseases, neoplastic disorders, inflammatory conditions, and conditions in which the sebaceous glands and/or the entire hair follicle are blocked.

14. The method according to claim 11, wherein the at least one lipophilic bioactive agent is a pharmaceutical agent selected from the group consisting of anti-inflammatory drugs, calcineurin inhibitors, Janus kinase inhibitors, 5-alpha reductase inhibitors, PDE4 inhibitors, immunomodulating agents, anti-viral agents, anti-fungal agents, antibiotics, steroids, prostaglandin analogues, apoptosis inhibitors, antiparasitic agents, anaesthetic agents, statins, anti-hyperlipidemia agents, anti-hypercholesterolemia agents and anti-cancer agents.

15. The method according to claim 14, wherein the at least one bioactive agent is finasteride and/or dutasteride.

16. The method according to claim 11, wherein following the application of the composition to the skin, the amount of the bioactive agent per unit area present in the hair follicles is higher than that the amount per unit area present in the skin of the subject being treated.

17. The method according to claim 11, wherein following the application of the composition to the skin, the concentration of the bioactive agent in the blood is lower than the concentration needed to achieve the desired systemic therapeutic effect.

18. The method according to claim 11, wherein following the administration of the composition to the skin, the concentration of the bioactive agent in the blood is at least 10 times lower than the concentration that would be obtained following oral or parenteral administration of a therapeutic dose of the same bioactive agent.
